(19) United States
(12) Patent Application Publication
(10) Pub. No.: US 2015/0150901 A1
(43) Pub. Date: Jun. 4, 2015

(54) Co-Therapy for Diabetic Conditions

(71) Applicant: Daiichi Sankyo, Inc., Parsippany, NJ (US)

(72) Inventor: Michael R. Jones, New York, NY (US)

(21) Appl. No.: 14/617,378

(22) Filed: Feb. 9, 2015

Related U.S. Application Data
(63) Continuation of application No. 14/011,224, filed on Aug. 27, 2013, now abandoned.
Application No. 14/011,224 is a continuation of application No. 13/584,874, filed on Aug. 14, 2012, now abandoned.
Application No. 13/584,874 is a continuation of application No. 12/816,653, filed on Jun. 16, 2010, now abandoned.
Application No. 12/816,653 is a continuation of application No. 11/446,053, filed on Jun. 2, 2006, now abandoned.
(60) Provisional application No. 60/702,895, filed on Jul. 27, 2005.

(51) Int. Cl. A61K 31/785; A61K 31/64; A61K 38/28; A61K 31/155

(52) U.S. Cl. CPC A61K 31/785; A61K 31/155; A61K 31/64; A61K 38/28

(57) ABSTRACT

Methods of treating diseases such as diabetes are disclosed. Methods of modulating elevated fructosamine levels, elevated HbAlc levels, impaired glucose tolerance, and impaired fasting glucose are also disclosed. In some embodiments, methods include co-administration of a bile acid sequestrant and two or more additional compounds selected from the group consisting of a biguanide, a sulfonylurea and insulin, or pharmaceutically acceptable salts thereof. Drug products including a bile acid sequestrant and two or more additional compounds selected from the group consisting of a biguanide, a sulfonylurea and insulin, or pharmaceutically acceptable salts thereof, in combination are also disclosed.

### CROSS-REFERENCE TO RELATED APPLICATION

This application is a continuation of U.S. patent application Ser. No. 14/011,224, filed Aug. 27, 2013, which is a continuation of U.S. patent application Ser. No. 13/584,874, filed Aug. 14, 2012, now abandoned, which is a continuation of U.S. patent application Ser. No. 12/816,653, filed Jun. 16, 2010, now abandoned, which is a continuation of U.S. patent application Ser. No. 11/446,053, filed on Jun. 2, 2006, now abandoned, which claims the benefit of U.S. Provisional Patent Application Ser. No. 60/702,895, filed on Jul. 27, 2005, all of which are incorporated herein by reference in their entireties.

### BACKGROUND OF THE INVENTION

The present invention generally relates to diabetic conditions and drug products for treatment of these conditions.

At least about 16 million Americans have type 2 diabetes. Individuals afflicted with type 2 and type 1 diabetes have elevated blood sugar levels due to problems with either the amount of or action of insulin, which regulates the body's handling of glucose. In type 1 diabetes, the pancreas is unable to respond normally to blood sugar levels by secreting insulin. In type 2 diabetes, the more common form, the liver and peripheral tissues may be less responsive to insulin. In later stages of type 2 diabetes, the pancreas may also secrete inadequate amounts of insulin for proper blood sugar control. Diabetic individuals who control blood glucose levels can substantially reduce the risk of developing vascular complications of diabetes, including, but not limited, to diabetic retinopathy (a condition which leads to blindness), diabetic nephropathy, diabetic neuropathy, and atherosclerosis.

The American Diabetes Association has recommended that patients with type 2 diabetes be treated to a goal of glycosylated hemoglobin A (HbAlc) of <7%, the level at which clinical trials have demonstrated fewer long-term microvascular complications. From the Third National Health and Nutrition Examination Survey data, it appears that only about 40% of patients with type 2 diabetes achieve this goal.

Taking care of patients with diabetes mellitus and its complications is estimated to cost more than $132 billion each year. Much of the personal and economic burden related to the care of diabetic patients stems from inadequate glycemic (blood glucose) control. Studies have demonstrated that glycemic control in the majority of patients with type 2 diabetes is inadequate. The position statement of the ADA recommends that all patients with type 2 diabetes be treated with diet, exercise, and when necessary, with medication to bring their HbAlc levels to below a threshold of 7%. An epidemiological analysis of the UK Prospective Diabetes Study data demonstrated an approximate 14% reduction in all-cause mortality and myocardial infarction for every 1% reduction in HbAlc. Furthermore, it is estimated that there is a 15%-30% reduction in the risk of microvascular complications for each 1% reduction in HbAlc.

Current pharmacologic methods of controlling blood glucose concentration include insulin injections or oral administration of sulfonylureas (e.g., glyburide), biguanide drugs (e.g., metformin), alpha-glucosidase inhibitors (e.g., acarbose), or thiazolidinediones (e.g., pioglitzone and rosiglitazone). Each of these therapies, however, suffers from various drawbacks, including treatment failure and undesirable adverse events. It would be desirable to improve current glucose lowering therapies, while minimizing undesirable adverse events.

### SUMMARY OF THE INVENTION

One aspect of the present invention pertains to methods for the treatment of diabetes and diabetic conditions and modulating elevated blood glucose levels, elevated fructosamine levels, elevated HbAlc levels, impaired glucose tolerance or impaired fasting glucose. According to one embodiment, treatment is effected by co-administering to a patient in need thereof therapeutically effective amounts of a bile acid sequestrant and two or more additional compounds selected from the group consisting of a biguanide, a sulfonylurea, and insulin, or pharmaceutically acceptable salts thereof. Specific embodiments include the administration of therapeutically effective amounts of colesevelam, metformin hydrochloride and glipizide or glyburide; colesevelam, metformin hydrochloride and insulin; and colesevelam, insulin, and glipizide or glyburide.

A second aspect of the present invention pertains to a drug product comprising a bile acid sequestrant and two or more additional active ingredients selected from the group consisting of a biguanide, a sulfonylurea, and insulin, or pharmaceutically acceptable salts thereof. In some embodiments, for example, when insulin is not included in the co-administration regimen, the drug product can be provided in a single dosage form or, alternatively, the drug product can be separated in a single container or package with instructions for co-administration as may be appropriate when insulin is part of the co-administration regimen.

### DETAILED DESCRIPTION

It is to be appreciated that the various method steps described herein include approximations of dosage amounts and may be varied. Before describing several exemplary embodiments of the invention, it is to be understood that the invention is not limited to the details set forth in the following description. The invention is capable of other embodiments and of being practiced or carried out in various ways.

In overview, one aspect of the invention pertains to methods for the treatment of diabetes. Treatment may be effected by the co-administration to a patient, for example, a human, of a bile acid sequestrant and two or more additional active ingredients selected from the group consisting of a biguanide, a sulfonylurea, and insulin, or pharmaceutically acceptable salts thereof. As used herein, co-administration means administering a dosage of each of the compounds within twelve hours of each other to the same patient or subject.

In one embodiment, a method of modulating blood glucose levels in a patient in need thereof is provided comprising co-administering to said patient a bile acid sequestrant and two or more additional active ingredients selected from the group consisting of a biguanide, a sulfonylurea, and insulin, or pharmaceutically acceptable salts thereof. Modulating blood glucose levels as used herein is understood to indicate maintaining glucose levels within clinically normal ranges or lowering elevated blood glucose levels to a more clinically desirable level or range.

In other embodiments, methods of modulating elevated fructosamine and/or HbAlc levels are provided, for example, in patients afflicted with type 2 diabetes or non-insulin dependent (NIDDM) diabetes mellitus.

In further embodiments, methods are provided for modulating impaired glucose tolerance or impaired fasting glucose in patients in need of such modulation. Impaired glucose tolerance is generally defined as two-hour glucose levels of 140 to 199 mg per dL (7.8 to 11.0 mmol per L) on the 75 g glucose tolerance test. Impaired fasting glucose is generally defined as glucose levels of 100 to 125 mg per dL (5.6 to 6.9 mmol per L) in fasting patients. Patients with impaired glucose tolerance or impaired fasting glucose have a significant risk of developing diabetes and are an important group for preventing diabetes.

In the present specification, the meaning of terms “active agent”, “active compound” or in some cases “compound” should be understood as equivalent.

According to one or more embodiments, the methods comprise administering to a patient in need thereof a co-therapy of (1) a therapeutically effective amount of a bile acid sequestrant, such as colesevelam (commercially known as Welchol®, cholestyramine (commercially known as Questran®, Cholybar®), colestipol (commercially known as Colestid®), or pharmaceutically acceptable salts thereof, and (2) a therapeutically effective amount of two or more additional compounds selected from the group consisting of a biguanide, such as metformin; a sulfonylurea, such as glipizide, glyburide, glimepiride, gliclazide, glibenclamide, gliquidone, acetohexamide, chlorpropamide, tolazamide and tolbutamide; and insulin; or pharmaceutically acceptable salts thereof.

As used herein, a pharmaceutically or therapeutically effective amount is understood to be at least a minimal amount which provides a medical improvement in the symptoms of the specific malady or disorder experienced by the mammal in question. Preferably, the recipient will experience a reduction, inhibition or removal of the biological basis for the malady in question.

A presently preferred bile acid sequestrant is colesevelam hydrochloride sold under the trademark Welchol® by Sankyo Pharma Inc. Generally the daily dosage colesevelam hydrochloride is between about 1.5 grams and 3.75 grams and usually does not exceed 3.75 grams per day.

A presently preferred salt of metformin is metformin hydrochloride, although the present invention is not limited to a particular salt. Metformin hydrochloride is commercially available in 500 mg, 850 mg and 1000 mg tablets from, e.g., Bristol Meyers Squibb under the Glucophage® trademark. Metformin hydrochloride may be administered in humans at an initial daily dose of from about 500 mg to 1000 mg and increased, as needed, to a maximum daily dosage of 2550 mg.

Presently preferred sulfonylureas include second generation sulfonylureas such as glipizide and glyburide. Glipizide is commercially available in 5 mg and 10 mg tablets from e.g., Pfizer under the Glucotrol® trademark. Glipizide may be administered in humans at an initial daily dose of from 2.5 mg to 5 mg and increased, as needed, to a maximum daily dosage of 40 mg. Glyburide is commercially available in 1.25 mg, 2.5 mg, and 5 mg tablets from e.g., Pfizer under the Diaβeta® trademark. Glipizide may be administered in humans at an initial daily dose of from 2.5 mg to 5 mg and increased, as needed, to a maximum daily dosage of 20 mg.

Various forms of animal and human insulin, both natural and recombinant, can be used in the methods of the invention, including rapid acting, short acting, intermediate acting and long lasting forms. These forms are commercially available under various trademarks, including Humulin® (Eli Lilly), Humalog® (Eli Lilly), Novolin® (Novo Nordisk), Novalog® (Novo Nordisk), Velosulin® (Novo Nordisk), and Lantus® (Aventis). The total individual insulin requirement is usually between 0.5 and 1.0 U/kg/day, but may be adjusted depending on the patient's needs.

Other dosages and dosage forms are within the scope of the present invention. The dosages may be varied depending upon the requirements of the patient, the severity of the condition being treated and the compound being employed. Determination of the proper dosage for a particular situation is within the skill of the art. In one embodiment, generally, treatment is initiated with smaller dosages which are less than the optimum dose of the compound. Thereafter, the dosage is increased by small increments until the optimum effect under the circumstance is reached.

It is understood that the dosage, regimen and mode of administration of these compounds will vary according to the condition and the individual being treated and will be subject to the judgment of the medical practitioner involved. It is preferred that the administration of one or more of the compounds herein begin at a low dose and be increased until the desired effects are achieved. It is also preferred that the recipient also utilize art recognized lifestyle patterns for reducing the incidence of the maladies described herein. These include maintenance of an appropriate diet and exercise regimen, as recommended by a medical practitioner familiar with the physical condition of the recipient.

While the individual compounds can be administered at different times, they also can be administered at the same time. When some or all of the agents are given substantially simultaneously, they may be given by a single fixed combination dosage form (for example, in embodiments that do not include insulin in the co-administration regimen) or by different dosage forms, whichever is convenient or necessary based on the properties of the drug. For example, metformin and glipizide can be administered simultaneously as the combination drug Metaglip® (Bristol Myers Squibb). Similarly, metformin and glyburide can be administered simultaneously as the combination drug Glucovance® (Bristol Myers Squibb). It will be appreciated that presently, insulin is administered parenterally and that colesevelam is administered orally. Accordingly, these drugs could not presently be combined in a single dosage form. However, it is envisioned within the scope of the present invention that if mutually compatible administration routes were developed, for example, if oral insulin dosages become available, co-administration in a single dosage form would be available.

When given in different dosage forms, it is irrelevant whether the route of administration is the same for each agent or different for each agent. Any route of administration known for the individual agents is acceptable for the practice of the present invention. The agents can be given in a fixed combination, or at least substantially simultaneously, i.e. within about 1 hour of each other. However, the agents can be administered at different times, and the invention benefits may still be realized. When administered at different times, it is believed that the agents should be given within about twelve hours of each other, preferably within about four hours of each other, and more preferably within about two hours of each other. Of course, these time periods can be adjusted if the dosage form is one which will “administer” the agents for extended periods.

Dosages of the agents include all dosages at which the agents are used individually as discussed above. The proper dosage for each agent can be obtained from any convenient reference such as the Physician's Desk Reference (PDR) or the label for each agent. Modified dosage ranges for mammals of varying sizes and stages of development will be apparent to those of ordinary skill.

It may be desirable if some or all of the agents are incorporated into a single dosage form such that the agents are physically separated. This may be accomplished in any of the myriad ways known in the art, such as bi-layered tablets, coated pellets of one agent incorporated into a tablet of the other, separately coated pellets of each agent in a capsule or tablet, coated pellets of one agent in capsule together with powder of the other agent, each agent microencapsulated separately and then blended together for use in a tablet or capsule, use of a dual or multiple compartment transdermal device, etc.

According to one or more embodiments of the invention, the preparation of pharmaceutical compositions can involve the use of pharmaceutically acceptable carriers, which can be either solid or liquid. Solid form preparations include powders, tablets, pills, capsules, sachets, suppositories, and dispersible granules. A solid carrier can be one or more substances which may also act as diluents, flavoring agents, solubilizers, lubricants, suspending agents, binders, preservatives, tablet disintegrating agents, an encapsulating material, or drug delivery agents, such as liposomal preparations.

One or more embodiments of the invention include solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

In embodiments including powders, the carrier typically is a finely divided solid which is in a mixture with the finely divided active component. In embodiments including tablets, the active component is mixed with the carrier having the necessary binding properties in suitable proportions and compacted in the shape and size desired.

Suitable carriers include, but are not limited to, magnesium carbonate, magnesium stearate, talc, sugar, lactose, pectin, dextrin, starch, gelatin, tragacanth, methyl cellulose, sodium carboxymethyl cellulose, a low melting wax, cocoa butter, and the like. The term “preparation” is intended to include the formulation of the active compound with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizing, and thickening agents as desired. Aqueous suspensions suitable for oral use can be made by dispersing the finely divided active component in water with viscous material, such as natural or synthetic gums, resins, methyl cellulose, sodium carboxymethyl cellulose, and other well-known suspending agents.

The pharmaceutical preparations are preferably in unit dosage form. In such form, the preparations are subdivided into unit doses containing appropriate quantities of the active component. The unit dosage form can be a packaged preparation, the package containing discrete quantities of preparation, such as packeted tablets, capsules, and powders in vials or ampules. Also, the unit dosage form can be a capsule, tablet, cachet, or lozenge itself, or it can be the appropriate number of any of these in packaged form.

The pharmaceutical preparations can be provided as a drug product comprising a bile acid sequestrant, a biguanide, and one or more additional active ingredients selected from the group consisting of a sulfonylurea and insulin. Two or more of the pharmaceutical preparations can be provided in the drug product as a combination single dosage form (when appropriate) and packaged with instructions for use, with the remaining pharmaceutical preparations (if any) included in a single container or package with the combination single dosage form with instructions for co-administration use.

Exemplary embodiments of the invention will be further described for illustrative purposes with reference to the following non-limiting examples.

### EXAMPLES

### Example 1

### Administration of Colesevelam to Diabetic Patients Taking Metformin, a Sulfonylurea or Both.

A prospective, randomized, double-blind, placebo-controlled, parallel group study, consisting of a 5-week, placebo run-in period (i.e., 1 week of screening and then 4 weeks of placebo treatment) followed by a 12-week active treatment period was conducted to determine the effects of a bile acid sequestrant on diabetic patients taking metformin, a sulfonylurea, or metformin and a sulfonylurea. Eligible patients were randomized to either WelChoi® (3.75 g/day in 6 tablets/day) or placebo (6 tablets/day). Enrollment was limited to patients with type 2 diabetes who were receiving a stable dose of treatment with a sulfonylurea, metformin, or the combination of metformin and a sulfonylurea, and whose glucose was not adequately controlled at a third visit (HbAlc 7.0% to 10.0%, inclusive). Patients who met the initial entry criteria were re-evaluated 4 weeks later to confirm the stability of their HbAlc measurement (i.e., did not differ from the initial screening value by more than 0.5%). Patients who met this criterion and the other entry criteria were then randomized to receive either WelChoi® (3.75 g/day in 6 tablets/day) or placebo (6 tablets/day) for 12 weeks.

As noted above, prior to entering the study, patients were receiving sulfonylurea, metformin, or the combination of metformin and a sulfonylurea. The dose of antidiabetic medication must have been stable for 90 days prior to Visit 1 (Week-5). All other antidiabetic agents were to be discontinued for at least 90 days prior to Visit 1 (Week-5). The use of any other investigational drug was prohibited. A total of 27 (41.5%) patients took a sulfonylurea alone, 9 (13.8%) patients took metformin, and 29 (44.6%) patients took the combination.

The primary efficacy parameter was the change in HbAlc from Week 0 (baseline) to Week 12. The secondary efficacy parameters included the change in HbAlc from baseline to Weeks 4 and 8; the change in fructosamine from baseline to Weeks 4, 8, and 12; the change in protein-bound glucose from baseline to Weeks 4, 8, and 12; the change in fasting plasma glucose level (FPG) from baseline to Weeks 4, 8, and 12; the change in meal glucose response from baseline to Weeks 1 and 12; the change in pre-prandial and postprandial glucose from baseline to Weeks 1 and 12; the change in free fatty acids from baseline to Weeks 4, 8, and 12; the change in insulin from baseline to Weeks 4, 8, and 12; and the change in homeostasis model assessment (HOMA) index from baseline to Weeks 4, 8, and 12. Percent changes in lipid parameters and changes in lipid subfractions were also evaluated.

The primary null hypothesis was that there was no difference between the treatment groups in the primary efficacy parameter, change in HbAlc from baseline to Week 12. When normality of the data was not violated, a mixed effect analysis of covariance (ANCOVA) model with treatment group as a fixed effect, center as a random effect, and the corresponding baseline value as a covariate was used. Least-squares (LS) mean, standard error, corresponding 95% confidence interval (CI), and p-value were calculated for the treatment difference. The approach used for analysis of the primary efficacy parameter was also applied to analysis of the secondary diabetic efficacy parameters. A mixed effect analysis of variance model with treatment group (fixed effect) and center (random effect) as factors was used to analyze the percent change in lipid parameters and change in lipid subfractions from baseline to Week 12.

Following 12 weeks of treatment, HbAlc was reduced by 0.3% in the WelChoi® group and increased by 0.2% in the placebo group. The LS mean treatment difference was statistically significant (−0.5%; p=0.007). A subgroup analysis demonstrated that for patients with HbAlc >8.0%, the mean change in HbAlc from baseline to Week 12 was −0.7% for the WelChol group and 0.2% for the placebo group; the LS mean treatment difference was statistically significant (−1.0%; p=0.002). A post-hoc subgroup analysis showed that for patients with HbAlc <7.5%, the LS mean change in HbAlc from baseline to Week 12 was −0.5% for the WelChoi® group and 0.2% for the placebo group; the LS mean treatment differences was statistically significant (−0.8%; p=0.001).

Following 12 weeks of treatment, fructosamine was reduced by 10.9 μmol/L in the WelChoi® group and increased by 11.7 μmol/L in the placebo group, yielding a statistically significant treatment difference (−29.0 μmol/L; p=0.011). Throughout the study, FPG was reduced to a greater extent in the WelChoi® group compared to the placebo group. Although the LS mean treatment difference in FPG was statistically significant at Week 4 (−23.3 mg/dL; p=0.016) and Week 8 (−18.3 mg/dL; p=0.011), the difference between the WelChoi® and the placebo treatment groups was not statistically significant at Week 12 (−14.0 mg/dL; p=0.118).

At Week 12, postprandial glucose was reduced by 17.8 mg/dL in the WelChoi® group compared to a 2.7 mg/dL increase in the placebo group; the LS mean difference between the treatment groups was statistically significant (−31.5 mg/dL; p=0.026). There were no statistically significant treatment differences with regard to changes from baseline in protein-bound glucose, meal glucose response, free fatty acids, insulin, or HOMA index.

Treatment with WelChoi® over a 12-week period resulted in statistically significant percent reductions in low-density lipoprotein cholesterol (−11.7%; p=0.007), total cholesterol (−7.3%; p=0.019), and apolipoprotein B (−11.8%; p=0.003) compared to placebo, and a statistically significant reduction in low-density lipoprotein particle concentration (−209.6 nmol/L; p=0.037) compared to placebo. There were no statistically significant treatment differences with regard to any other lipid parameters.

Treatment with WelChoi® for 12 weeks resulted in statistically significant reductions in HbAlc, fructosamine, and postprandial glucose compared to treatment with placebo. These results demonstrate that a bile acid sequestrant such as WelChoi® may be a useful agent for improving glycemic control in patients with type 2 diabetes mellitus who are on metformin therapy or metformin therapy in combination with another agent, such as a sulfonylurea. According to the present invention other bile acid sequestrants may be administered to patients receiving metformin therapy alone or metformin therapy combined with another drug. Addition of WelChoi® to treatment did not result in any new, unexpected safety or tolerability issues.

The treatment difference in HbAlc of 0.5% reduction in the WelChoi® group compared to the placebo group is both highly statistically significant (p=0.007) and clinically meaningful. The importance of the reduction in HbAlc is further demonstrated by a treatment difference in HbAlc levels of −0.8% and −1.0% in the subgroups of patients with a baseline HbAlc ≧7.5% (p=0.001) and ≧8.0% (p=0.002), respectively. The results of these subgroup analyses suggest that WelChoi®, when used as an agent to improve glycemic control, may be more useful in patients who are most in need of additional drug support (i.e., those individuals with higher HbAlc levels and already on metformin therapy alone or in combination with another drug).

In addition to the beneficial effects observed in the diabetic parameters, WelChoi® also reduced LDL-C, LDL particle concentration, Total-C, and apo B levels. Compared to placebo, treatment with WelChoi® over a 12-week period resulted in statistically significant mean percent reductions in LDL-C (−11.7%; p=0.007), Total-C (−7.3%; p=0.019), and apo B (−11.8%; p=0.003), and a statistically significant mean reduction in LDL particle concentration (−209.6 nmol/L; p=0.037). The improvement in both glycemic and lipid parameters contributes to a reduction in the global risk for coronary heart disease in these high-risk patients with diabetes.

### Example 2

### Administration of Colesevelam to Diabetic Patients Taking Metformin.

A prospective, randomized, double-blind, placebo-controlled, parallel group study, consisting of a 3-week, placebo run-in period (i.e., 1 week of screening and then 2 weeks of placebo treatment) followed by a 26-week active treatment period is conducted to confirm the glucose-lowering effect of colesevelam on type 2 diabetic patients taking metformin seen in Example 1. The study randomizes ˜300 patients to either colesevelam (3.8 g/day) or placebo and has a 81% to >95% power to detect a difference of 0.54% to 0.80% between colesevelam and placebo in mean HbAlc reductions from baseline with a 2-sided type I error at 0.05 assuming a common standard deviation of at most 1.5% and a maximum dropout rate of 15%.

The population for the study is males and females between the ages of 18 and 75, inclusive, not adequately controlled on metformin monotherapy or metformin in combination with other oral anti-diabetic agents for the treatment of type 2 diabetes, with HbAlc ≧7.5% and ≦9.5%. Patients are on their stable dose of metformin for at least 90 days prior to the study. Patients remain on their stable dose of metformin and any other oral anti-diabetic agent (as defined as the dose taken during the screening period) and do not change the dose for the duration of the study. If during the treatment period a patient's HbAlc increases to ≧10.0%, the patient is discontinued from the study.

At the completion of the run-in period, patients who meet the entry criteria are assigned randomly on a 1:1 basis to either colesevelam in the form of WelChoi® 3.8 g/day (in 6 tablets/day) (˜150 patients) or placebo (˜150 patients). Patients take their metformin and any other oral anti-diabetic medication at the same time it was taken prior to the start of the study.

Patients are evaluated and blood samples drawn at weeks 6, 12, 18 and 26 of the double-blind treatment period. The primary efficacy variable is the change in plasma HbAlc from baseline to week 26 endpoint. The secondary efficacy variables include the change in HbAlc from baseline to weeks 6, 12 and 18; changes in FPG and fructosamine from baseline to weeks 6, 12, 18 and week 26 endpoint; the glycemic control response rate (defined as a reduction in FPG of ≧30 mg/dL or a reduction in HbAlc of ≧0.7% from baseline to week 26 endpoint); insulin sensitivity (e.g., changes in insulin, C-peptide, HOMA, and adiponectin from baseline to week 26 endpoint); change in high-sensitivity C-reactive protein (hsCRP) from baseline to week 26 endpoint; and percent changes in lipids (e.g., LDL-C, non-HDL-C, TG, TC, HDL-C, apo A-I, and apo B) from baseline to week 26 endpoint. The primary null hypothesis is that there is no difference between the treatment groups in the primary efficacy parameter, change in HbAlc from baseline to Week 26 endpoint. Baseline is defined as the last measurement prior to the first dose of randomized study medication.

A mixed-effects ANCOVA model with treatment as a fixed effect, center as a random effect, and baseline as a covariate is used to test the primary null hypothesis. The treatment difference in HbAlc change from baseline to week 26 endpoint between WelChoi® and placebo is evaluated by the LS mean, standard error, the 2-tailed 95% CI and the 2-sided p-value. The treatment-by-center interaction and treatment-by-covariate interaction is evaluated for the primary efficacy variable at a significance level of 0.10. If a significant treatment-by-center interaction and treatment-by-covariate interaction is suggested by the data, further analyses are implemented to assess the qualitative or quantitative nature of the interaction. The approach used for analysis of the primary efficacy variable is also applied to analysis of the secondary efficacy variables.

### Example 3

### Administration of Colesevelam to Diabetic Patients Taking a Sulfonylurea.

A prospective, randomized, double-blind, placebo-controlled, parallel group study, consisting of a 3-week, placebo run-in period (i.e., 1 week of screening and then 2 weeks of placebo treatment) followed by a 26-week active treatment period is conducted to confirm the glucose-lowering effect colesevelam on type 2 diabetic patients taking a sulfonylurea seen in Example 1. The study randomizes ˜400 patients to either colesevelam (3.8 g/day) or placebo and has a 86% to 95% power to detect a difference of 0.50% to 0.80% between colesevelam and placebo in mean HbAlc reductions from baseline with a 2-sided type I error at 0.05 assuming a common standard deviation of at most 1.5% and a maximum dropout rate of 15%.

The population for the study is males and females between the ages of 18 and 75, inclusive, not adequately controlled on sulfonylurea monotherapy or sulfonylurea in combination with other oral anti-diabetic agents for the treatment of type 2 diabetes (e.g., metformin, thiazolidinedione), with HbAlc ≧7.5% and ≦9.5%. Patients are on their stable dose of sulfonylurea for at least 90 days prior to the study. Patients remain on their stable dose of sulfonylurea and any other oral anti-diabetic agent (as defined as the dose taken during the screening period) and do not change the dose for the duration of the study. If during the treatment period a patient's HbAlc increases to ≧10.0%, the patient is discontinued from the study.

At the completion of the run-in period, patients who meet the entry criteria are assigned randomly on a 1:1 basis to either colesevelam in the form of WelChoi® 3.8 g/day (in 6 tablets/day) (˜200 patients) or placebo (˜200 patients). Patients take their sulfonylurea and any other oral anti-diabetic medication at the same time it was taken prior to the start of the study.

Patients are evaluated and blood samples drawn at weeks 6, 12, 18 and 26 of the double-blind treatment period. The primary efficacy variable is the change in plasma HbAlc from baseline to week 26 endpoint. The secondary efficacy variables include the change in HbAlc from baseline to weeks 6, 12 and 18; changes in FPG and fructosamine from baseline to weeks 6, 12, 18 and week 26 endpoint; the glycemic control response rate (defined as a reduction in FPG of ≧30 mg/dL or a reduction in HbAlc of ≧0.7% from baseline to week 26 endpoint); insulin sensitivity (e.g., changes in insulin, C-peptide, HOMA, and adiponectin from baseline to week 26 endpoint); change in high-sensitivity C-reactive protein (hsCRP) from baseline to week 26 endpoint; and percent changes in lipids (e.g., LDL-C, non-HDL-C, TG, TC, HDL-C, apo A-I, and apo B) from baseline to week 26 endpoint. The primary null hypothesis is that there is no difference between the treatment groups in the primary efficacy parameter, change in HbAlc from baseline to Week 26 endpoint. Baseline is defined as the last measurement prior to the first dose of randomized study medication.

A mixed-effects ANCOVA model with treatment as a fixed effect, center as a random effect, and baseline as a covariate is used to test the primary null hypothesis. The treatment difference in HbAlc change from baseline to week 26 endpoint between WelChoi® and placebo is evaluated by the LS mean, standard error, the 2-tailed 95% CI and the 2-sided p-value. The treatment-by-center interaction and treatment-by-covariate interaction is evaluated for the primary efficacy variable at a significance level of 0.10. If a significant treatment-by-center interaction and treatment-by-covariate interaction is suggested by the data, further analyses are implemented to assess the qualitative or quantitative nature of the interaction. The approach used for analysis of the primary efficacy variable is also applied to analysis of the secondary efficacy variables. To further evaluate the efficacy results, change in HbAlc from baseline to week 26 endpoint is summarized by four subgroups of patients: patients on sulfonylurea alone, patients on sulfonylurea and metformin, patients on sulfonylurea and thiazolidinedione, and patients on unspecified sulfonylurea/oral anti-diabetic combinations. ANCOVA model is used to analyze the data for each of the four subgroups, with treatment as a fixed effect and baseline HbAlc as a covariate.

### Example 4

### Administration of Colesevelam to Diabetic Patients Taking Insulin.

A prospective, randomized, double-blind, placebo-controlled, parallel group study, consisting of a 3-week, placebo run-in period (i.e., 1 week of screening and then 2 weeks of placebo treatment) followed by a 16-week active treatment period is conducted to investigate the effect of colesevelam on type 2 diabetic patients taking insulin. The study randomizes ˜260 patients to either colesevelam (3.8 g/day) or placebo and has a 81% to 95% power to detect a difference of 0.58% to 0.80% between colesevelam and placebo in mean HbAlc reductions from baseline with a 2-sided type I error at 0.05 assuming a common standard deviation of at most 1.5% and a maximum dropout rate of 15%.

The population for the study is males and females between the ages of 18 and 75, inclusive, not adequately controlled on insulin monotherapy or insulin in combination with other oral anti-diabetic agents for the treatment of type 2 diabetes (e.g., metformin, sulfonylurea, thiazolidinedione), with HbAlc ≧7.5% and ≦9.5%. Patients are on their stable dose of insulin or insulin in combination other oral anti-diabetic agents for at least 6 weeks or 90 days, respectively, prior to the study. Patients remain on their stable dose of insulin and any other oral anti-diabetic agent (as defined as the dose taken during the screening period) and do not change the dose for the duration of the study. If during the treatment period a patient's HbAlc increases to ≧10.0%, the patient is discontinued from the study.

At the completion of the run-in period, patients who meet the entry criteria are assigned randomly on a 1:1 basis to either colesevelam in the form of WelChoi® 3.8 g/day (in 6 tablets/day) (˜130 patients) or placebo (˜130 patients). Patients take their sulfonylurea and any other oral anti-diabetic medication at the same time it was taken prior to the start of the study.

Patients are evaluated and blood samples drawn at weeks 4, 8, and 16 of the double-blind treatment period. The primary efficacy variable is the change in plasma HbAlc from baseline to week 16 endpoint. The secondary efficacy variables include the change in HbAlc from baseline to weeks 4 and 8; changes in FPG and fructosamine from baseline to weeks 4, 8 and week 26 endpoint; the glycemic control response rate (defined as a reduction in FPG of ≧30 mg/dL or a reduction in HbAlc of ≧0.7% from baseline to week 16 endpoint); changes C-peptide and adiponectin from baseline to week 16 endpoint; change in high-sensitivity C-reactive protein (hsCRP) from baseline to week 16 endpoint; and percent changes in lipids (e.g., LDL-C, non-HDL-C, TG, TC, HDL-C, apo A-I, and apo B) from baseline to week 16 endpoint. The primary null hypothesis is that there is no difference between the treatment groups in the primary efficacy parameter, change in HbAlc from baseline to Week 26 endpoint. Baseline is defined as the last measurement prior to the first dose of randomized study medication.

A mixed-effects ANCOVA model with treatment as a fixed effect, center as a random effect, and baseline as a covariate is used to test the primary null hypothesis. The treatment difference in HbAlc change from baseline to week 16 endpoint between WelChoi® and placebo is evaluated by the LS mean, standard error, the 2-tailed 95% CI and the 2-sided p-value. The treatment-by-center interaction and treatment-by-covariate interaction is evaluated for the primary efficacy variable at a significance level of 0.10. If a significant treatment-by-center interaction and treatment-by-covariate interaction is suggested by the data, further analyses are implemented to assess the qualitative or quantitative nature of the interaction. The approach used for analysis of the primary efficacy variable is also applied to analysis of the secondary efficacy variables. To further evaluate the efficacy results, change in HbAlc from baseline to week 16 endpoint is summarized by five subgroups of patients: patients on insulin alone, patients on insulin and sulfonylurea, patients on insulin and metformin, patients on insulin and thiazolidinedione, and patients on unspecified insulin/oral anti-diabetic combinations or insulin with combinations of oral anti-diabetic medications. ANCOVA model is used to analyze the data for each of the four subgroups, with treatment as a fixed effect and baseline HbAlc as a covariate.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed:

1. A method for treating diabetes in a human in need of such treatment, the method comprising administering to said human therapeutically effective amounts of a bile acid sequestrant and two or more additional compounds selected from the group consisting of a biguanide, a sulfonylurea, insulin, and pharmaceutically acceptable salts thereof.

2. The method of claim 1, wherein the bile acid sequestrant and the two or more compounds are administered substantially simultaneously.

3. The method of claim 1, wherein the bile acid sequestrant and the two or more compounds are administered separately.

4. The method of claim 2, wherein the bile acid sequestrant and the two or more compounds are administered within one hour of each other.

5. The method of claim 3, wherein the bile acid sequestrant and the two or more compounds are administered within twelve hours of each other.

6. The method of claim 1, wherein the bile acid sequestrant is selected from the group consisting of colesevelam, cholestyramine, and colestipol; the biguanide comprises metformin; and the sulfonylurea is selected from the group consisting of glipizide, glyburide glimepiride, gliclazide, glibenclamide, gliquidone, acetohexamide, chlorpropamide, tolazamide, and tolbutamide.

7. The method of claim 6, wherein the pharmaceutically acceptable salt of the bile acid sequestrant comprises colesevelam hydrochloride; the pharmaceutically acceptable salt of the biguanide comprises metformin hydrochloride; and the sulfonylurea comprises glipizide or glyburide.

8. The method of claim 1, wherein the two additional compounds are a biguanide and a sulfonylurea.

9. The method of claim 8, wherein the pharmaceutically acceptable salt of the bile acid sequestrant comprises colesevelam hydrochloride; the pharmaceutically acceptable salt of the biguanide comprises metformin hydrochloride; and the sulfonylurea comprises glipizide or glyburide.

10. The method of claim 1, wherein the two additional compounds are a biguanide and insulin.

11. The method of claim 10, wherein the pharmaceutically acceptable salt of the bile acid sequestrant comprises colesevelam hydrochloride and the pharmaceutically acceptable salt of the biguanide comprises metformin hydrochloride.

12. The method of claim 1, wherein the two additional compounds are a sulfonylurea and insulin.

13. The method of claim 12, wherein the pharmaceutically acceptable salt of the bile acid sequestrant comprises colesevelam hydrochloride and the sulfonylurea comprises glipizide or glyburide.

14. A method for modulating a condition in a human in need of such modulation, the condition selected from the group consisting of elevated blood glucose levels, elevated fructosamine levels, elevated HbAlc levels, impaired glucose tolerance, and impaired fasting glucose comprising co-administering to said subject therapeutically effective amounts of a bile acid sequestrant and two or more additional compounds selected from the group consisting of a biguanide, a sulfonylurea, insulin, and pharmaceutically acceptable salts thereof.

15. The method of claim 14, wherein the bile acid sequestrant is selected from the group consisting of colesevelam, cholestyramine, and colestipol; the biguanide comprises metformin; and the sulfonylurea is selected from the group consisting of glipizide, glyburide glimepiride, gliclazide, glibenclamide, gliquidone, acetohexamide, chlorpropamide, tolazamide, and tolbutamide.

16. The method of claim 15, wherein the pharmaceutically acceptable salt of the bile acid sequestrant comprises colesevelam hydrochloride; the pharmaceutically acceptable salt of the biguanide comprises metformin hydrochloride; and the sulfonylurea comprises glipizide or glyburide.

17. A drug product comprising a bile acid sequestrant and two or more additional active ingredients selected from the group consisting of a biguanide, a sulfonylurea, insulin, and pharmaceutically acceptable salts thereof.

18. The drug product of claim 17, wherein two or more of the ingredients are a combination single dosage.

19. The drug product of claim 18, wherein any remaining ingredients are included in a single container or package with the combination single dosage form with instructions for co-administration use.

20. The drug product of claim 17, wherein the bile acid sequestrant is selected from the group consisting of colesevelam, cholestyramine, and colestipol; the biguanide comprises metformin; and the sulfonylurea is selected from the group consisting of glipizide, glyburide glimepiride, gliclazide, glibenclamide, gliquidone, acetohexamide, chlorpropamide, tolazamide, and tolbutamide.

21. The drug product of claim 20, wherein the pharmaceutically acceptable salt of the bile acid sequestrant comprises colesevelam hydrochloride; the pharmaceutically acceptable salt of the biguanide comprises metformin hydrochloride; and the sulfonylurea comprises glipizide or glyburide.
